(19) United States
(12) Patent Application Publication
(10) Pub. No.: US 2001/0044107 A1
(43) Pub. Date: Nov. 22, 2001

(54) Recombinase mediated gene chip detection

(76) Inventors: David A. Zarling, Menlo Park, CA (US); Gurucharan Reddy, Fremont, CA (US); Sushma Pati, Woodside, CA (US)

Correspondence Address: FLEHR HOHBACH TEST, ALBRITTON & HERBERT LLP, Four Embarcadero Center - Suite 3400, San Francisco, CA, 94111-4187, US

(21) Appl. No.: 09/751,671

(22) Filed: Dec. 28, 2000

Related U.S. Application Data
(60) Provisional application No. 60/173,348, filed on Dec. 28, 1999.

(51) Int. Cl. C12Q 1/68; C12N 11/16

(52) U.S. Cl. 435/6; 435/174; 438/1

(57) ABSTRACT

The present invention is directed to the use of recombinases such as E. coli recA to mediate the detection of target sequences on gene chips.

This application is a continuing application of U.S. Ser. No. 60/173,348 filed Dec. 28, 1999, hereby expressly incorporated by reference.

### FIELD OF THE INVENTION

The present invention is directed to the use of recombinases such as E. coli RecA protein to mediate the detection of target sequences on gene chips.

### BACKGROUND OF THE INVENTION

The detection of specific nucleic acids is an important tool for diagnostic medicine and molecular biology research. Gene probe assays currently play roles in identifying infectious organisms such as bacteria and viruses, in probing the expression of normal and mutant genes and identifying mutant genes such as oncogenes, in typing tissue for compatibility preceding tissue transplantation, in matching tissue or blood samples for forensic medicine, and for measuring homology among genes from different species.

Currently, there are several types of types of gene microarray technologies with arrayed DNA sequences of known identity; these include arraying cDNA on a substrate and the immobilization of oligonucleotide probes. In either version, the gene chips are exposed to DNA or RNA targets, generally single stranded, to allow for hybridization between the immobilized probe and the target. Watson-Crick DNA-DNA hybridization is the basic underlying principle for both of these microarray formats and thus native target nucleic acid is always denatured for use in these microarray formats. The DNA-DNA hybridization is a non-enzymatic mass action driven process dependent on reaction time, temperature and DNA concentration which can result in a number of hybridization reactions and artifacts, including incorrect sequence alignments due to repeat sequences in DNA. An additional problem with mass action based DNA-DNA hybridization procedures is the presence of secondary structures in single-stranded DNA substrates in single-stranded DNA substrates which can severely affect the hybridization process and lead to either misleading results or those that are hard to interpret.

RecA protein (or its homologues such as Rad51) binds to either single-stranded DNA or RNA to form right handed helical structures known as nucleoprotein filaments. RecA protein binds to single-stranded DNA in a cooperative manner and stretches the DNA approximately 1.5 times the length of the B-form of DNA and in the process removes the secondary structures in the single-stranded DNA or RNA. These nucleoprotein filaments rapidly catalyze the search for homology to find a homologous or partly homologous native non-denatured DNA target in a vast excess of genomic or other gene sequences. Depending on the conditions, RecA nucleoprotein filaments allow native DNA hybridization with either completely homologous DNA or with DNA containing significant heterologies (up to 30% mismatch). This is important for mutation detection and gene family detection. 10 Accordingly, it is an object of the present invention to provide methods of facilitating the use of gene chips by using recombinase.

### SUMMARY OF THE INVENTION

In accordance with the objects outlined above, the present invention provides compositions comprising a substrate comprising an array of capture probes, at least one of which comprises a recombinase, and are preferably coated with recombinase. The recombinase can be a RecA recombinase such as E. coli RecA, a RecA peptide, a thermostable RecA, a Rad51 recombinase, etc.

In a further aspect, the capture probes are covalently attached to said substrate and may comprise DNA.

In an additional aspect, the invention provides methods of detecting the presence of a target sequence in a sample comprising providing a substrate comprising an array of capture probes, contacting the target sequence with the array, wherein either the capture probes or the target sequence is coated with a recombinase, to form an assay complex. The presence or absence of the assay complex is then detected as an indication of the presence of the target sequence. The target sequence can be either RNA or DNA.

### DETAILED DESCRIPTION OF THE INVENTION

The present invention is directed to the use of recombinases in the detection of nucleic acid sequences using gene chips. There are a wide variety of known gene chips comprising nucleic acid capture probes that are used to detect nucleic acid sequences, and the addition of a recombinase can increase specificity and augment hybridization kinetics. The system can be used in one of two ways; either the recombinase is coated onto the soluble target sequences, which are then added to an array, or the recombinase can be on the capture probes on the solid support (added either pre- or post array synthesis). The present invention finds use in a wide variety of assays, including gene expression profiling, nucleic acid diagnostic assays, genotyping, etc. as is further described below.

RecA nucleoprotein filaments can also be used to efficiently catalyze the homologous recognition reaction with homologous or homoeologous (partially homologous) native dsDNA fragments or large genomic DNA on gene chips. Gene chip based homologous recognition has significant commercial applications in the arena of gene chip technology for massively parallel processing and high throughput gene analysis, mutant gene detection and gene expression analysis. Gene chip based homologous and homeologous gene recognition also has significant applications in gene discovery, drug discovery, pharmacogenomics and toxicology research.

Accordingly, the present invention provides compositions and methods for detecting and/or quantifying nucleic acids, such as target nucleic acid sequences, in a sample. As will be appreciated by those in the art, the sample solution may comprise any number of things, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples; purified samples, such as purified genomic DNA, RNA, proteins, etc.; raw samples (bacteria, virus, genomic DNA, etc.; As will be appreciated by those in the art, virtually any experimental manipulation may have been done on the sample.

The present invention provides compositions and methods for detecting the presence or absence of target nucleic acid sequences in a sample. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of labels, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribonucleotides, and any combination of bases, including uracil, adenine, thymine, cytosine, guanine, inosine, xathanine hypoxathanine, isocytosine, isoguanine, etc. A preferred embodiment utilizes isocytosine and isoguanine in nucleic acids designed to be complementary to other probes, rather than target sequences, as this reduces non-specific hybridization, as is generally described in U.S. Pat. No. 5,681,702. As used herein, the term “nucleoside” includes nucleotides as well as nucleoside and nucleotide analogs, and modified nucleosides such as amino modified nucleosides. In addition, “nucleoside” includes non-naturally occuring analog structures. Thus for example the individual units of a peptide nucleic acid, each containing a base, are referred to herein as a nucleoside.

The compositions and methods of the invention are directed to the detection of target sequences. The term “target sequence” or “target nucleic acid” or grammatical equivalents herein means a nucleic acid sequence generally on a single strand of nucleic acid (although as will be appreciated by those in the art, the present invention can utilize double stranded targets as well, or targets that comprise both single stranded portions and double stranded portions). The target sequence may be a portion of a gene, a regulatory sequence, genomic DNA, cDNA, RNA including mRNA and rRNA, or others. As is outlined herein, the target sequence may be a target sequence from a sample, or a secondary target such as a product of a reaction such as a PCR or other amplification reaction, etc. Thus, for example, a target sequence from a sample is amplified to produce a secondary target that is detected; alternatively, an amplification step is done using a signal probe that is amplified, again producing a secondary target that is detected. The target sequence may be any length, with the understanding that longer sequences are more specific. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others. As is outlined more fully below, capture probes are made to hybridize to target sequences to determine the presence, absence or quantity of a target sequence in a sample. Generally speaking, this term will be understood by those skilled in the art. The target sequence may also be comprised of different target domains; for example, in “sandwich” type assays as outlined herein, a first target domain of the sample target sequence may hybridize to a capture probe and a second target domain may hybridize to a portion of a label probe, etc. In addition, the target domains may be adjacent (i.e. contiguous) or separated. For example, when oligonucleotide ligation assay (OLA) techniques are used, a first primer may hybridize to a first target domain and a second primer may hybridize to a second target domain; either the domains are adjacent, or they may be separated by one or more nucleotides, coupled with the use of a polymerase and dNTPs, as is more fully outlined below. The terms “first” and “second” are not meant to confer an orientation of the sequences with respect to the 5′−3′ orientation of the target sequence. For example, assuming a 5′−3′ orientation of the complementary target sequence, the first target domain may be located either 5′ to the second domain, or 3′ to the second domain. In addition, as will be appreciated by those in the art, the probes on the surface of the array (e.g. the capture probes) may be attached in either orientation, either such that they have a free 3′ end or a free 5′ end; in some embodiments, the probes can be attached at one ore more internal positions, or at both ends.

If required, the target sequence is prepared using known techniques. For example, the sample may be treated to lyse the cells, using known lysis buffers, sonication, electroporation, etc., with purification and amplification occurring as needed, as will be appreciated by those in the art. In addition, the reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents which may be included in the assays. These include reagents like salts, buffers, neutral proteins, e.g. albumin, detergents, etc., which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target.

In a preferred embodiment, amplification of the target sequence is done prior to detection. As will be appreciated by those in the art, there are a wide variety of suitable amplification techniques. Suitable amplification methods include both target amplification and signal amplification and include, but are not limited to, polymerase chain reaction (PCR), ligation chain reaction (sometimes referred to as oligonucleotide ligase amplification OLA), cycling probe technology (CPT), strand displacement assay (SDA), transcription mediated amplification (TMA), nucleic acid sequence based amplification (NASBA), rolling circle amplification (RCA), and invasive cleavage technology. In addition, there are a number of variations of PCR which also may find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, “reverse transcriptase PCR” or “RT-PCR”, “biotin capture PCR”, “vectorette PCR”. “panhandle PCR”, and “PCR select cDNA subtration”, among others. All of these methods require a primer nucleic acid (including nucleic acid analogs) that is hybridized to a target sequence to form a hybridization complex, and an enzyme is added that in some way modifies the primer to form a modified primer. For example, PCR generally requires two primers, dNTPs and a DNA polymerase; LCR requires two primers that adjacently hybridize to the target sequence and a ligase; CPT requires one cleavable primer and a cleaving enzyme; invasive cleavage requires two primers and a cleavage enzyme; etc. Thus, in general, a target nucleic acid is added to a reaction mixture that comprises the necessary amplification components, and a modified primer is formed which is then detected as outlined below.

As required, the unreacted primers are removed, in a variety of ways, as will be appreciated by those in the art. The hybridization complex is then disassociated, and the modified primer is detected and optionally quantitated on an array as outlined herein. In some cases, the newly modified primer serves as a target sequence for a secondary reaction, which then produces a number of amplified strands, which can be detected as outlined herein.

In addition, in some embodiments, double stranded target nucleic acids are denatured to render them single stranded so as to permit hybridization of the primers and other probes of the invention. A preferred embodiment utilizes a thermal step, generally by raising the temperature of the reaction to about 95° C., although pH changes and other techniques may also be used. However, as outlined herein, one significant advantage of the present invention is that when the capture probes comprise the recombinase, the target sequences need not be denatured. RecA also tolerates double stranded nucleic acids and heterologies (mismatches).

The target sequences can be labeled for detection in a variety of ways, as will be appreciated by those in the art. A variety of labeling techniques can be done. In general, either direct or indirect detection of the target products can be done. “Direct” detection as used in this context, as for the other reactions outlined herein, requires the incorporation of a label, in this case a detectable label, preferably an optical label such as a fluorophore, into the target sequence, with detection proceeding as outlined below. In this embodiment, the label(s) may be incorporated in a variety of ways: (1) the primers comprise the label(s), for example attached to the base, a ribose, a phosphate, or to analogous structures in a nucleic acid analog; (2) modified nucleosides are used that are modified at either the base or the ribose (or to analogous structures in a nucleic acid analog) with the label(s); these label-modified nucleosides are then converted to the triphosphate form and are incorporated into a newly synthesized strand by a polymerase; or (3) a label probe that is directly labeled and hybridizes to a portion of the target sequence can be used. Any of these methods result in a newly synthesized strand or reaction product that comprises labels, that can be directly detected as outlined below.

Thus, the modified strands comprise a detection label, that may be a primary label or a secondary label. Accordingly, detection labels may be primary labels (i.e. directly detectable) or secondary labels (indirectly detectable).

In a preferred embodiment, the detection label is a primary label. A primary label is one that can be directly detected, such as a fluorophore. In general, labels fall into three classes: a) isotopic labels, which may be radioactive or heavy isotopes; b) magnetic, electrical, thermal labels; and c) colored or luminescent dyes. Labels can also include enzymes (horseradish peroxidase, etc.) and magnetic particles. Preferred labels include chromophores or phosphors but are preferably fluorescent dyes. Suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, quantum dots (also referred to as “nanocrystals”: see U.S. Ser. No. 09/315,584, hereby incorporated by reference), pyrene, Malacite green, stilbene, Lucifer Yellow, Cascade Blue™, Texas Red, Cy dyes (Cy3, Cy5, etc.), alexa dyes, phycoerythin, bodipy, and others described in the 6th Edition of the Molecular Probes Handbook by Richard P. Haugland, hereby expressly incorporated by reference.

In a preferred embodiment, a secondary detectable label is used. A secondary label is one that is indirectly detected; for example, a secondary label can bind or react with a primary label for detection, go or can act on an additional product to generate a primary label (e.g. enzymes). Secondary labels include, but are not limited to, one of a binding partner pair; chemically modifiable moieties; nuclease inhibitors, enzymes such as horseradish peroxidase, alkaline phosphatases, lucifierases, etc.

In a preferred embodiment, the secondary label is a binding partner pair. For example, the label may be a hapten or antigen, which will bind its binding partner. In a preferred embodiment, the binding partner can be attached to a solid support to allow separation of extended and non-extended primers. For example, suitable binding partner pairs include, but are not limited to: antigens (such as proteins (including peptides)) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid-nucleic acid binding proteins pairs are also useful. In general, the smaller of the pair is attached to the NTP for incorporation into the primer. Preferred binding partner pairs include, but are not limited to, biotin and streptavidin, digeoxinin and Abs, and Prolinx™ reagents (see www.prolinxinc.com/ie4home.hmtl).

In a preferred embodiment, the binding partner pair comprises a primary detection label (for example, attached to the NTP and therefore to the extended primer) and an antibody that will specifically bind to the primary detection label. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁻⁴-10⁻⁶M⁻¹, with less than about 10⁻⁵ to 10⁻⁹M⁻¹ being preferred and less than about 10⁻⁷-10⁻¹ being particularly preferred.

The target sequences (again, optionally labeled) are added to an array of capture probes. The present system finds particular utility in array formats, i.e. wherein there is a matrix of addressable microscopic locations(herein generally referred to “pads”, “addresses” or “micro-locations”). The size of the array will depend on the composition and end use of the array. Nucleic acids arrays are known in the art, and can be classified in a number of ways; both ordered arrays (e.g. the ability to resolve chemistries at discrete sites), and random arrays are included. Ordered arrays include, but are not limited to, those made using photolithography techniques (Affymetrix GeneChip™), spotting techniques (Synteni and others), printing techniques (Hewlett Packard and Rosetta), three dimensional “gel pad” arrays, bead arrays, etc.

Arrays containing from about 2 different capture probes to many millions can be made, with very large arrays being possible. Generally, the array will comprise from two to as many as a billion or more, depending on the size of the addresses and the substrate, as well as the end use of the array. Preferred ranges for the arrays range from about 100 to about 100,000 addresses per square centimeter. In addition, due to the extra “size” of the recombinases used herein, it may be desirable to lower the density of probes at any particular address.

In some embodiments, the compositions of the invention may not be in array format; that is, for some embodiments, substrates comprising a single capture probe may be made as well. In addition, in some arrays, multiple substrates may be used, either of different or identical compositions. Thus for example, large arrays may comprise a plurality of smaller substrates.

The capture probes of the invention are designed to be complementary to a target sequence such that hybridization of the target sequence and the probes of the present invention occurs. This complementarity need not be perfect; there may be any number of base pair mismatches which will interfere with hybridization between the target sequence and the capture probes of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

The size of the probe may vary, as will be appreciated by those in the art, in general varying from 5 to 500 nucleotides in length, with probes of between 10 and 100 being preferred, between 15 and 50 being particularly preferred, and from 20 to 35 being especially preferred.

The arrays of the invention comprise a substrate to which the capture probes are immobilized. By “substrate” or “solid support” or other grammatical equivalents herein is meant any material that can be used to immobilize nucleic acids and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, optical fiber bundles, and a variety of other polymers. In general, the substrates allow optical detection and do not themselves appreciably fluoresce.

Generally the substrate is flat (planar), although as will be appreciated by those in the art, other configurations of substrates may be used as well; for example, three dimensional configurations can be used, for example by embedding the capture probes in a porous block of plastic that allows sample access to the probes and using a confocal microscope for detection. Similarly, the capture probes may be placed on the inside surface of a tube, for flow-through sample analysis to minimize sample volume.

The capture probes can be immobilized to the substrate in a wide variety of ways, as is known in the art. Generally, the substrate is functionalized to include a reactive group that can be used to immobilize (generally through covalent attachment, but not always) the capture probes. In many cases the capture probe is synthesized using standard techniques, and includes a functional group that will react with the functional group on the substrate.

As outlined herein, one of the components of the hybridization complexes comprises a recombinase. As will be appreciated by those in the art, the systems of the invention can take on a number of different configurations, depending on the type of array, the assay, and the end use of the array. For example, when “direct” assays are run, that is, where the target sequence is directly hybridized to the capture probe, either the capture probe or the target sequence may be coated with the recombinase. Alternatively, when “sandwich” type assays are run, and assay complexes are formed that comprise at least the capture probe, the target sequence, and a label probe, any one of the components of the assay complex can comprise the recombinase.

Thus, one of the nucleic acids of the invention are coated with recombinase. “Recombinase” refers to a family of RecA-like recombination proteins all having essentially all or most of the same functions, particularly: (i) the recombinase protein's ability to properly bind to and position a probe to it's homologous target and (ii) the ability of recombinase protein/polynucleotide complexes to efficiently find and bind to complementary endogenous sequences. The best characterized RecA protein is from the bacterium E. coli. In addition to the wild-type protein a number of mutant RecA proteins have been identified (e.g., RecA803; see Madiraju et al., PNAS USA 85(18):6592 (1988); Madiraju et al, Biochem. 31:10529 (1992); Lavery et al., J. Biol. Chem. 267:20648 (1992)). Further, many organisms have RecA-like recombinases with strand-transfer activities (e.g., Fugisawa et al., (1985) Nucl. Acids Res. 13: 7473; Hsieh et al., (1986) Cell 44: 885; Hsieh et al., (1989) J. Biol. Chem. 264: 5089; Fishel et al., (1988) Proc. Natl. Acad. Sci. (USA) 85: 3683; Cassuto et al., (1987) Mol. Gen. Genet. 208: 10; Ganea et al., (1987) Mol. Cell Biol. 7: 3124; Moore et al., (1990) J. Biol. Chem. 19: 11108; Keene et al., (1984) Nucl. Acids Res. 12: 3057; Kimeic, (1984) Cold Spring Harbor Svmp. 48: 675; Kmeic, (1986) Cell 44: 545; Kolodner et al., (1987) Proc. Natl. Acad. Sci. USA 84: 5560; Sugino et al., (1985) Proc. Natl. Acad. Sci. USA 85: 3683; Halbrook et al., (1989) J. Biol. Chem. 264: 21403; Eisen et al., (1988) Proc. NatI. Acad. Sci. USA 85: 7481; McCarthy et al., (1988) Proc. Natl. Acad. Sci. USA 85: 5854; Lowenhaupt et al., (1989) J. Biol. Chem. 264: 20568, which are incorporated herein by reference). Examples of such recombinase proteins include, for example but not limited to: RecA, RecA803, UvsX, and other RecA mutants and RecA-like recombinases (Roca, A. l. (1990) Crit. Rev. Biochem. Molec. Biol. 25: 415), sep1 (Kolodner et al. (1987) Proc. Natl. Acad. Sci. (U.S.A.) 84:5560; Tishkoff et al. Molec. Cell. Biol. 11:2593), RuvC (Dunderdale et al. (1991) Nature 354: 506), DST2, KEM1, XRN1 (Dykstra et al. (1991) Molec. Cell. Biol. 11:2583), STP/DST1 (Clark et al. (1991) Molec. Cell. Biol. 11:2576), HPP-1 (Moore et al. (1991) Proc. Natl. Acad. Sci. (U.S.A.) 88:9067), other target recombinases (Bishop et al. (1992) Cell 69: 439; Shinohara et al. (1992) Cell 69: 457); incorporated herein by reference). RecA may be purified from E. coli strains, such as E. coli strains JC12772 and JC15369 (available from A. J. Clark and M. Madiraju, University of California-Berkeley, or purchased commercially). These strains contain the recA coding sequences on a “runaway” replicating plasmid vector (present at a high copy number in the cell). The RecA803 protein is a high-activity mutant of wild-type RecA. The art teaches several examples of recombinase proteins, for example, from Drosophila, yeast, plant, human, and non-human mammalian cells, including proteins with biological properties similar to RecA (i.e., RecA-like recombinases), such as Rad51 (including Rad51A, B, C and D, XRCC2 and XRCC3), Rad57, Dmc from mammals and yeast, hereby incorporated by reference). In addition, the recombinase may actually be a complex of proteins, i.e. a “recombinosome”. In addition, included within the definition of a recombinase are portions or fragments of recombinases which retain recombinase biological activity, as well as variants or mutants of wild-type recombinases which retain biological activity, such as the E. coli RecA803 mutant with enhanced recombinase activity or recombinases such as RecA that have been shuffled or altered to increase activity or for other reasons.

In a preferred embodiment, RecA or a Rad51 is used, including the RecA peptide (sometimes referred to herein as FECO peptide; see U.S. Pat. No. 5,731,411, hereby expressly incorporated by reference), and thermostabile RecA. For example, RecA protein is typically obtained from bacterial strains that overproduce the protein: wild-type E. coli RecA protein and mutant RecA803 protein may be purified from such strains. Alternatively, RecA protein can also be purchased from, for example, Pharmacia (Piscataway, N.J.) or Boehringer Mannheim (Indianapolis, Ind.).

RecA proteins, and their homologs, form a nucleoprotein filament when they coat a single-stranded DNA molecule. In this nucleoprotein filament, one monomer of RecA protein is bound to about 3 nucleotides. This ability of RecA to coat single-stranded DNA is essentially sequence independent, although particular sequences favor initial loading of RecA onto a polynucleotide (e.g., nucleation sequences). The nucleoprotein filament(s) can be formed on essentially any DNA molecule and can be formed in cells (e.g., mammalian cells), forming complexes with both single-stranded and double-stranded DNA, although the loading conditions for dsDNA are different than for ssDNA.

The nucleic acids of the invention are coated with recombinase. The conditions used to coat targeting polynucleotides with recombinases such as recA protein and ATPyS have been described in commonly assigned U.S. Ser. No. 07/910,791, filed Jul. 9, 1992; U.S. Ser. No. 07/755,462, filed Sep. 4, 1991; and U.S. Ser. No. 07/520,321, filed May 7, 1990, each incorporated herein by reference. The procedures below are directed to the use of E. coli recA, although as will be appreciated by those in the art, other recombinases may be used as well. Targeting polynucleotides can be coated using GTPyS, mixes of ATPyS with rATP, rGTP and/or dATP, or dATP or rATP alone in the presence of an rATP generating system (Boehringer Mannheim). Various mixtures of GTPyS, ATPyS, ATP, ADP, dATP and/or rATP or other nucleosides may be used, particularly preferred are mixes of ATPyS and ATP or ATPyS and ADP.

RecA protein coating of targeting polynucleotides is typically carried out as described in U.S. Ser. No. 07/910,791, filed Jul. 9, 1992 and U.S. Ser. No. 07/755,462, filed Sep. 4, 1991, which are incorporated herein by reference. Briefly, the targeting polynucleotide, whether double-stranded or single-stranded, is denatured by heating in an aqueous solution at 95-100° C. for five minutes, then placed in an ice bath for 20 seconds to about one minute followed by centrifugation at 0° C. for approximately 20 sec, before use. When denatured targeting polynucleotides are not placed in a freezer at −200° C. they are usually immediately added to standard recA coating reaction buffer containing ATPYS, at room temperature, and to this is added the recA protein. Alternatively, recA protein may be included with the buffer components and ATPyS before the polynucleotides are added.

RecA coating of targeting polynucleotide(s) is initiated by incubating polynucleotide-recA mixtures at 37° C. for 10-15 min. RecA protein concentration tested during reaction with polynucleotide varies depending upon polynucleotide size and the amount of added polynucleotide, and the ratio of recA molecule:nucleotide preferably ranges between about 3:1 and 1:3. When single-stranded polynucleotides are recA coated independently of their homologous polynucleotide strands, the mM and μM concentrations of ATPyS and recA, respectively, can be reduced to one-half those used with double-stranded targeting polynucleotides (i.e., recA and ATPyS concentration ratios are usually kept constant at a specific concentration of individual polynucleotide strand, depending on whether a single- or double-stranded polynucleotide is used).

RecA protein coating of targeting polynucleotides is normally carried out in a standard 1× RecA coating reaction buffer. 10× RecA reaction buffer (i.e., 10× AC buffer) consists of: 100 mM Tris acetate (pH 7.5 at 37° C.), 20 mM magnesium acetate, 500 mM sodium acetate, 10 mM DTT, and 50% glycerol). All of the targeting polynucleotides, whether double-stranded or single-stranded, typically are denatured before use by heating to 95-100° C. for five minutes, placed on ice for one minute, and subjected to centrifugation (10,000 rpm) at 0° C. for approximately 20 seconds (e.g., in a Tomy centrifuge). Denatured targeting polynucleotides usually are added immediately to room temperature RecA coating reaction buffer mixed with ATPyS and diluted with double-distilled H₂O as necessary.

A reaction mixture typically contains the following components: (i) 0.2-4.8 mM ATPyS; and (ii) between 1-100 ng/μl of targeting polynucleotide. To this mixture is added about 1-20 μl of recA protein per 10-100 μl of reaction mixture, usually at about 2-10 mg/ml (purchased from Pharmacia or purified), and is rapidly added and mixed. The final reaction volume-for RecA coating of targeting polynucleotide is usually in the range of about 10-500 μl. RecA coating of targeting polynucleotide is usually initiated by incubating targeting polynucleotide-RecA mixtures at 37° C. for about 10-15 min.

RecA protein concentrations in coating reactions varies depending upon targeting polynucleotide size and the amount of added targeting polynucleotide: recA protein concentrations are typically in the range of 5 to 50 μM. When single-stranded targeting polynucleotides are coated with recA, independently of their complementary strands, the concentrations of ATPyS and recA protein may optionally be reduced to about one-half of the concentrations used with double-stranded targeting polynucleotides of the same length: that is, the recA protein and ATPyS concentration ratios are generally kept constant for a given concentration of individual polynucleotide strands.

The coating of targeting polynucleotides with recA protein can be evaluated in a number of ways. First, protein binding to DNA can be examined using band-shift gel assays (McEntee et al., (1981) J. Biol. Chem. 256: 8835). Labeled polynucleotides can be coated with recA protein in the presence of ATPyS and the products of the coating reactions may be separated by agarose gel electrophoresis. Following incubation of recA protein with denatured duplex DNAs the recA protein effectively coats single-stranded targeting polynucleotides derived from denaturing a duplex DNA. As the ratio of recA protein monomers to nucleotides in the targeting polynucleotide increases from 0, 1:27, 1:2.7 to 3.7:1 for 121-mer and 0, 1:22, 1:2.2 to 4.5:1 for 159-mer, targeting polynucleotide's electrophoretic mobility decreases, i.e., is retarded, due to recA-binding to the targeting polynucleotide. Retardation of the coated polynucleotide's mobility reflects the saturation of targeting polynucleotide with recA protein. An excess of recA monomers to DNA nucleotides is required for efficient recA coating of short targeting polynucleotides (Leahy et al., (1986) J. Biol. Chem. 261: 954).

A second method for evaluating protein binding to DNA is in the use of nitrocellulose fiber binding assays (Leahy et al., (1986) J. Biol. Chem. 261:6954; Woodbury, et al., (1983) Biochemistry 22(20):4730-4737. The nitrocellulose filter binding method is particularly useful in determining the dissociation-rates for protein:DNA complexes using labeled DNA. In the filter binding assay, DNA:protein complexes are retained on a filter while free DNA passes through the filter. This assay method is more quantitative for dissociation-rate determinations because the separation of DNA:protein complexes from free targeting polynucleotide is very rapid.

As outlined herein, the systems of the invention can take on a number of configurations. In a preferred embodiment, the target sequences comprise the recombinase. In this embodiment, the target sequences are prepared as needed, and then coated with the recombinase as outlined herein.

Alternatively, in a preferred embodiment, the capture probes on the substrate comprise the recombinase. In a preferred embodiment, for example when the arrays are made using techniques that take full length capture probes and attach them to the substrate, for example in spotting or printing techniques, the recombinase can be added either before or after attachment to the substrate. In a preferred embodiment, the capture probes are made and attached to the substrate, and then a recombinase is added to the array to coat the individual capture probes. Alternatively, a preferred embodiment utilizes a coating reaction prior to addition to the substrate.

In embodiments that rely on the use of arrays made by synthesizing the capture probes directly on the surface, such as those that rely on photolithographic techniques, the recombinase is preferably added to the capture probes after synthesis.

In addition, it should be noted that in some embodiments, for example in “sandwich” type assays, it is possible to have one or more of the components coated with recombinase. For example, some sandwich assays use a capture probe hybridized to a first portion of the target sequence, and a label probe that carries a detectable label and hybridizes to a second portion of the target sequence. In this case, it may be the capture probe, the target sequence, the label probe, or any combination that carries the recombinase.

The target sequences are added to the array of capture probes under conditions suitable for the formation of hybridization complexes. A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology--Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of helix destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Thus, the assays are generally run under stringency conditions which allows formation of the hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The sample comprising the target sequences and the array comprising the capture probes (one of which comprises the recombinase) are added together under conditions that allow the formation of hybridization complexes. Detection proceeds in a wide variety of ways, depending on the label and density of the array. Usually, when fluorescent labels are used, optical detectors such as CCD cameras or confocal microscopes are used. In addition, a number of other components can be present, such as CPUs or other processors, keyboards, ports, etc. to allow for detection and quantification.

Once made, the compositions find use in a wide variety of applications. As is known in the art, there are a wide variety of nucleic acid assays in use currently, and thus the methods and compositions of the present invention may be used in a variety of research, clinical, quality control, or field testing settings, including nucleic acid diagnostic assays, gene expression profiling, genotyping including single nucleotide polymorphism (SNP) detection, sequencing by hybridization, etc.

In a preferred embodiment, the probes are used in genetic diagnosis. For example, probes can be made using the techniques disclosed herein to detect target sequences such as the gene for nonpolyposis colon cancer, the BRCA1 breast cancer gene, p53, which is a gene associated with a variety of cancers, the Apo E4 gene that indicates a greater risk of Alzheimer's disease, allowing for easy presymptomatic screening of patients, mutations in the cystic fibrosis gene, or any of the others well known in the art, including mutations such as SNPs.

In an additional embodiment, viral and bacterial detection is done using the complexes of the invention. In this embodiment, probes are designed to detect target sequences from a variety of bacteria and viruses. For example, current blood-screening techniques rely on the detection of anti-HIV antibodies. The methods disclosed herein allow for direct screening of clinical samples to detect HIV nucleic acid sequences, particularly highly conserved HIV sequences. In addition, this allows direct monitoring of circulating virus within a patient as an improved method of assessing the efficacy of anti-viral therapies. Similarly, viruses associated with leukemia, HTLV-I and HTLV-II, may be detected in this way. Bacterial infections such as tuberculosis, clymidia and other sexually transmitted diseases, may also be detected.

In a preferred embodiment, the nucleic acids of the invention find use as probes for toxic bacteria in the screening of water and food samples. For example, samples may be treated to lyse the bacteria to release its nucleic acid, and then probes designed to recognize bacterial strains, including, but not limited to, such pathogenic strains as, Salmonella, Campylobacter, Vibrio cholerae, Leishmania, enterotoxic strains of E. coli, and Legionnaire's disease bacteria. Similarly, bioremediation strategies may be evaluated using the compositions of the invention.

In a further embodiment, the probes are used for forensic “DNA fingerprinting” to match crime-scene DNA against samples taken from victims and suspects.

In an additional embodiment, the probes in an array are used for sequencing by hybridization.

In a preferred embodiment, the arrays are used for mRNA detection and gene expression profiling as is well known in the art. In particular, RecA and other recombinases are known to bind to RNA, and thus RNA-coated with recombinases can be added to arrays for direct gene expression profiling.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### RecA Mediated Homologous Recognition on Gene Chips for Detection of Differential Gene Expression in Normal versus Tumor Cells

cDNA or genomic DNA is immobilized on a gene chip, RecA coated mRNA fragments mediate homologous recognition on the solid surface without any denaturation and allow the determination of differential gene expression in cancer cells compared to normal cells. The expression pattern of Rad51 and its homologues, Rad51 B, C, D XRCC2, XRCC3 and DMC1 from normal fibroblast cells are to be compared with the expression pattern in a breast tumor cell line. RNA is extracted from both the normal and tumor cell lines and labeled either directly with fluorescent tags or amplified and then labeled (one example of a good amplification technique for RNA is to reverse transcribe the RNA to cDNA and then label during transcription). The labeled RNA is fragmented and coated with RecA protein to make the nucleoprotein filaments and reacted with gene chips containing known cDNA clones at known locations. After targeting, unreacted RNA is washed away and the gene chip is exposed to illumination to record the intensities of the color at each spot and analyzed by a computer.

We claim:

1. A composition comprising a substrate comprising an array of capture probes, at least one of which comprises a recombinase.

2. A composition according to claim 1 wherein a plurality of said probes are coated with a recombinase.

3. A composition according to claim 1 or 2 wherein said recombinase is a RecA recombinase.

4. A composition according to claim 3 wherein said RecA recombinase is E. coli RecA.

5. A composition according to claim 3 wherein said RecA recombinase is RecA peptide.

6. A composition according to claim 1 wherein said recombinase is a Rad51 recombinase.

7. A composition according to claim 1 wherein said capture probes are covalently attached to said substrate.

8. A composition according to claim 1 wherein said capture probes comprise DNA.

9. A method of detecting the presence of a target sequence in a sample comprising:
is a) providing a substrate comprising an array of capture probes;
b) contacting said target sequence with said array, wherein either said capture probes or said target sequence is coated with a recombinase, to form an assay complex; and
c) detecting the presence of said assay complex as an indication of the presence of said target sequence.

10. A method according to claim 9 wherein said recombinase is a recA recombinase.

11. A method according to claim 10 wherein said recA recombinase is E. coli recA.

12. A method according to claim 9 wherein said capture probes comprise said recombinase.

13. A method according to claim 9 wherein said target sequence comprises said recombinase.

14. A method according to claim 13 further comprising coating said target sequence with said recombinase.

15. A method according to claim 9 wherein said target sequence is RNA.

16. A method according to claim 15 wherein said RNA is coated with a recombinase.
